(19) United States
(12) Patent Application Publication
(10) Pub. No.: US 2025/0303322 A1
(43) Pub. Date: Oct. 2, 2025

(54) METHODS FOR THE PRODUCTION OF CANNABINOID COMPOSITIONS

(71) Applicant: BUZZELET DEVELOPMENT AND TECHNOLOGIES LTD., Or Akiva (IL)

(72) Inventor: Aharon EYAL, Jerusalem (IL)

(21) Appl. No.: 18/864,526

(22) PCT Filed: May 9, 2023
(86) PCT No.: PCT/IB2023/054774

§ 371 Date: Nov. 10, 2024

Related U.S. Application Data
(60) Provisional application No. 63/341,236, filed on May 12, 2022.

(51) Int. Cl. B01D 15/24; B01D 15/18; C07C 37/00; C07C 37/68; C07C 37/82; C07D 311/80

(52) U.S. Cl. CPC B01D 15/24; B01D 15/1814; C07C 37/004; C07C 37/685; C07C 37/82; C07D 311/80; C07C 2601/16

(57) ABSTRACT

A method for direct current (DC)-DC conversion. The method includes converting an input voltage to a set of output voltages by selecting a first output capacitor in a set of output capacitors of a DC-DC converter, charging an inductor of the DC-DC converter, and discharging an electric current passing through the inductor into the first output capacitor. The first output capacitor is selected responsive to a voltage level of the first output capacitor being less than a first reference voltage in a set of reference voltages. The inductor is charged by applying the input voltage to the inductor. The first output capacitor maintains a first output voltage in the set of output voltages.

### TECHNICAL FIELD

The present disclosure generally relates to power supply methods, and particularly, to direct current (DC)-DC conversion.

### BACKGROUND ART

Function-rich system-on-chip applications require multiple power supplies to optimize overall system performances as well as to minimize power consumption. Direct current (DC)-DC converters are widely used for converting a power supply to multiple power supplies. Among various types of converters, LC (inductor-capacitor) DC-DC converters that utilize a capacitor and an inductor for each voltage output are a popular choice. However, drawbacks such as electromagnetic interference, component cost, and physical profile may prevent utilizing multiple inductors. Single inductor multiple-output (SIMO) DC-DC converters may be appropriate in integrated circuit (IC) applications since they may be area-and cost-effective, and each output voltage may have an adjustable conversion ratio.

A conventional SIMO DC-DC converter may generate multiple supply voltages. For each output, a respective output capacitor may be needed as a voltage storage element that maintains an output voltage. Also, an inductor may operate as a current storage element that transfers energy from input voltage to output voltages. To regulate each output voltage, an inductor current may be charged first, and then discharged in one of output capacitors to regulate corresponding voltage.

A conventional SIMO DC-DC converter may include a number of output switches that connect a shared node of an inductor to different output capacitors. Therefore, a shared node of an inductor may include a high parasitic capacitance, resulting in a high dynamic power dissipation. More power may be dissipated due to on-state resistance of output switches. On-state resistance of output switches may be smaller for larger output switches. However, larger output switches may include higher parasitic capacitance. For a fixed parasitic capacitance, dynamic power dissipation may increase with a switching frequency of output switches.

Therefore, a switching frequency may need to be made as small as possible for a minimal power dissipation.

Voltage levels of output voltages may be maintained by periodically charging output capacitors through output switches. Since different loads may consume different amount of power, a periodic switching may result in cross-regulation between different outputs. In other words, changes in a load of one output may change voltage levels of other outputs.

In pulse-width modulation (PWM) converters such as U.S. Pat. Nos. 5,617,015A and 8,624,429B2, voltage outputs may be maintained by increasing a duty cycle of switching an output switch to compensate for increased power consumption of a corresponding load. However, since switching frequency of PWM converters is fixed, increasing a duty cycle for one output switch may decrease duty cycles of switching other output switches. As a result, other outputs may be prone to instability.

There is, therefore, a need for a DC-DC voltage conversion method that switches different outputs as few as possible in a non-periodic manner.

### SUMMARY OF THE DISCLOSURE

This summary is intended to provide an overview of the subject matter of the present disclosure, and is not intended to identify essential elements or key elements of the subject matter, nor is it intended to be used to determine the scope of the claimed implementations. The proper scope of the present disclosure may be ascertained from the claims set forth below in view of the detailed description below and the drawings.

In one general aspect, the present disclosure describes an exemplary method for direct current (DC)-DC conversion. An exemplary method may include converting an input voltage to a set of output voltages by selecting a first output capacitor in a set of output capacitors of a DC-DC converter, charging an inductor of the DC-DC converter, and discharging an electric current passing through the inductor into the first output capacitor. In an exemplary embodiment, the first output capacitor may be selected utilizing one or more processors. An exemplary first output capacitor may be selected responsive to a voltage level of the first output capacitor being less than a first reference voltage in a set of reference voltages. An exemplary inductor may be charged utilizing the one or more processors. An exemplary inductor may be charged by applying the input voltage to the inductor. An exemplary electric current may be discharged utilizing the one or more processors. An exemplary first output capacitor may maintain a first output voltage in the set of output voltages.

In an exemplary embodiment, selecting the first output capacitor may include generating a set of request signals and selecting a first request signal in the set of request signals by prioritizing the set of request signals. Generating an exemplary set of request signals may include generating each request signal in the set of request signals responsive to a voltage level of a respective output capacitor in the set of output capacitors being less than a respective reference voltage in the set of reference voltages. An exemplary first request signal may be associated with the first output capacitor.

Selecting an exemplary first output capacitor may further include setting the set of request signals to an empty set. An exemplary set of request signals may be set to an empty set responsive to a consumed energy of a voltage source being higher than an input energy threshold. An exemplary voltage source may be associated with the input voltage.

Selecting an exemplary first output capacitor may further include eliminating an overcurrent request signal from the set of request signals. An exemplary overcurrent request signal may be eliminated responsive to a transferred energy from the voltage source to an overcurrent output capacitor in the set of output capacitors being higher than an output energy threshold.

Applying an exemplary input voltage to the inductor may include applying the input voltage to a first node of the inductor and coupling a second node of the inductor to a ground node. An exemplary input voltage may be coupled to the first node through a first switch of the DC-DC converter. An exemplary second node may be coupled to the ground node through a second switch of the DC-DC converter.

Discharging an exemplary electric current may include coupling the first node to the ground node, decoupling the second node from the ground node, and coupling the second node to the first output capacitor. An exemplary first node may be coupled to the ground node through a third switch of the DC-DC converter. An exemplary first node may be coupled to the ground node responsive to the DC-DC converter operating in a buck mode. An exemplary second node may be decoupled from the ground node by turning off the second switch. An exemplary second node may be coupled to the first output capacitor through an output switch of the DC-DC converter.

Discharging an exemplary electric current may further include decoupling the second node from the first output capacitor. An exemplary second node may be decoupled from the first output capacitor by turning off the output switch. An exemplary output switch may be turned off responsive to a value of the electric current being in a neighborhood of zero.

Discharging an exemplary electric current may further include decoupling the second node from the first output capacitor and coupling the first node to the second node. An exemplary second node may be decoupled from the first output capacitor by turning off the output switch. An exemplary output switch may be turned off responsive to a value of the electric current being in a neighborhood of a positive threshold. In an exemplary embodiment, coupling the first node to the second node may include turning on a fourth switch of the DC-DC converter. An exemplary fourth switch may be connected in parallel with the inductor.

Other exemplary systems, methods, features and advantages of the implementations will be, or will become, apparent to one of ordinary skill in the art upon examination of the following figures and detailed description. It is intended that all such additional systems, methods, features and advantages be included within this description and this summary, be within the scope of the implementations, and be protected by the claims herein.

### BRIEF DESCRIPTION OF THE DRAWINGS

The drawing figures depict one or more implementations in accord with the present teachings, by way of example only, not by way of limitation. In the figures, like reference numerals refer to the same or similar elements.

FIG. 1A shows a flowchart of a method for DC-DC conversion, consistent with one or more exemplary embodiments of the present disclosure.

FIG. 1B shows a flowchart of a method for selecting an output capacitor in a set of output capacitors, consistent with one or more exemplary embodiments of the present disclosure.

FIG. 1C shows a flowchart of a method for applying an input voltage to an inductor, consistent with one or more exemplary embodiments of the present disclosure.

FIG. 1D shows a flowchart of a method for applying an input voltage to an inductor, consistent with one or more exemplary embodiments of the present disclosure.

FIG. 1E shows a flowchart of a method for discharging an electric current into an output capacitor, consistent with one or more exemplary embodiments of the present disclosure.

FIG. 2A shows a schematic of a DC-DC converter, consistent with one or more exemplary embodiments of the present disclosure.

FIG. 2B shows a schematic of a DC-DC converter including a voltage divider and a comparator, consistent with one or more exemplary embodiments of the present disclosure.

FIG. 2C shows a schematic of a digital circuit, consistent with one or more exemplary embodiments of the present disclosure.

FIG. 3A shows control signals, an electric current, and output voltages for non-overlapped request signals, consistent with one or more exemplary embodiments of the present disclosure.

FIG. 3B shows control signals, an electric current, and output voltages for overlapped request signals, consistent with one or more exemplary embodiments of the present disclosure.

FIG. 3C shows an output voltage provided to a constant load, consistent with one or more exemplary embodiments of the present disclosure.

FIG. 3D shows a spectrum of an output voltage, consistent with one or more exemplary embodiments of the present disclosure.

FIG. 3E shows an output voltage provided to a variant load, consistent with one or more exemplary embodiments of the present disclosure.

FIG. 4 shows a high-level functional block diagram of a computer system, consistent with one or more exemplary embodiments of the present disclosure.

### DESCRIPTION OF EMBODIMENTS

In the following detailed description, numerous specific details are set forth by way of examples in order to provide a thorough understanding of the relevant teachings. However, it should be apparent that the present teachings may be practiced without such details. In other instances, well known methods, procedures, components, and/or circuitry have been described at a relatively high-level, without detail, in order to avoid unnecessarily obscuring aspects of the present teachings.

The following detailed description is presented to enable a person skilled in the art to make and use the methods and devices disclosed in exemplary embodiments of the present disclosure. For purposes of explanation, specific nomenclature is set forth to provide a thorough understanding of the present disclosure. However, it will be apparent to one skilled in the art that these specific details are not required to practice the disclosed exemplary embodiments. Descriptions of specific exemplary embodiments are provided only as representative examples. Various modifications to the exemplary implementations will be readily apparent to one skilled in the art, and the general principles defined herein may be applied to other implementations and applications without departing from the scope of the present disclosure. The present disclosure is not intended to be limited to the implementations shown, but is to be accorded the widest possible scope consistent with the principles and features disclosed herein.

Herein is disclosed an exemplary method for direct current (DC)-DC conversion. An exemplary method may include selecting an output capacitor of a DC-DC converter that requires charging to maintain an output voltage. An exemplary output capacitor may be selected when a voltage level of the output capacitor is smaller than a reference voltage of the output capacitor. When voltage levels of two or more output capacitors are less than respective reference voltages, the output capacitors may be prioritized to select an output capacitor with highest priority, that is, the output capacitor that is going to be charged first. After selecting an exemplary output capacitor, an inductor of the DC-DC converter may be charged by applying an input voltage to the inductor. Next, an electric current passing through the inductor may be discharged into the output capacitor with highest priority. To avoid drawing an excessive current from an exemplary output capacitor, transferred energy from a voltage source of the DC-DC converter to the output capacitor may be calculated, and when the transferred energy is larger than a threshold, the output capacitor may not be selected for charging. Similarly, to avoid drawing excessive current from the voltage source, a total transferred energy from the voltage source to all of output capacitors may be calculated, and when the total transferred energy is larger than a threshold, none of output capacitors may be selected. As a result, the transferred energy may decrease and the voltage source may be protected from overcurrent.

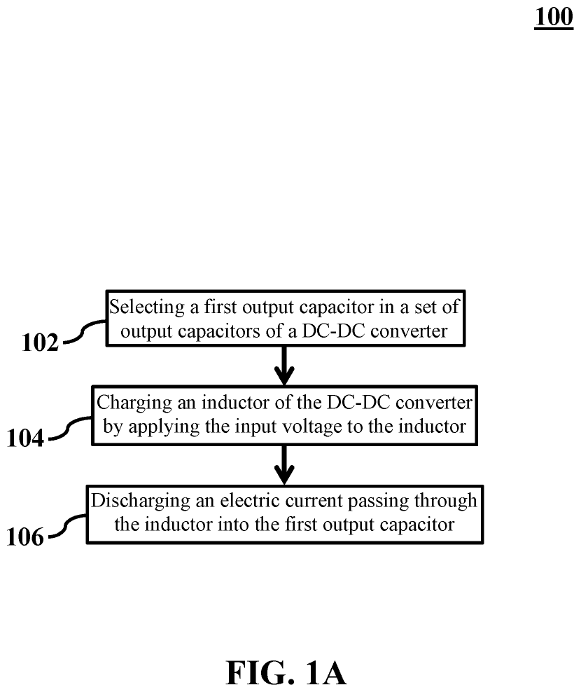

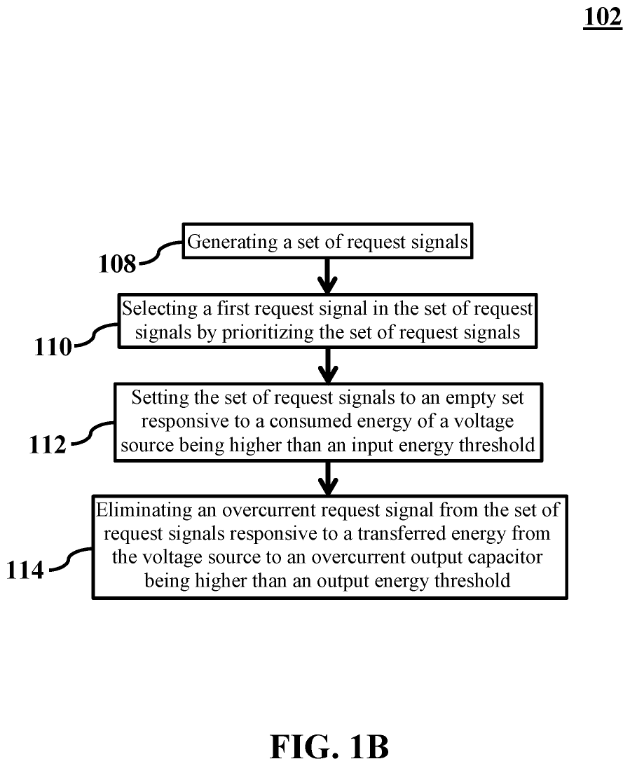

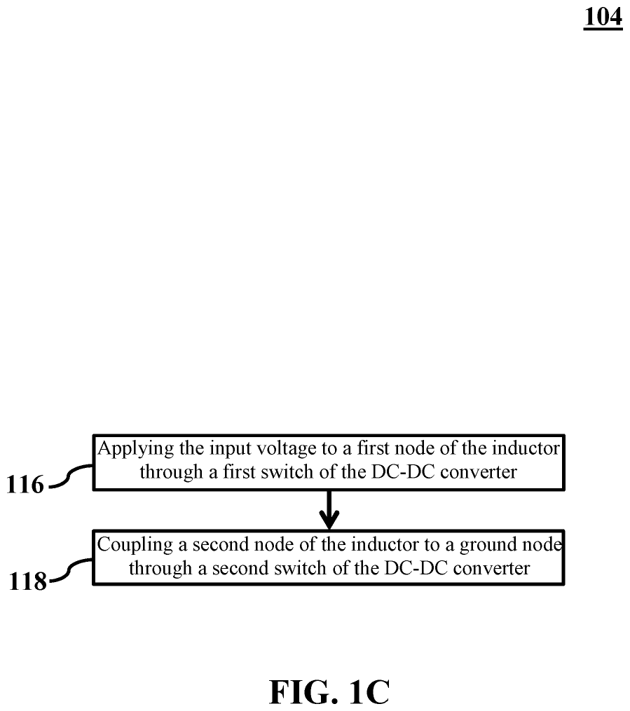

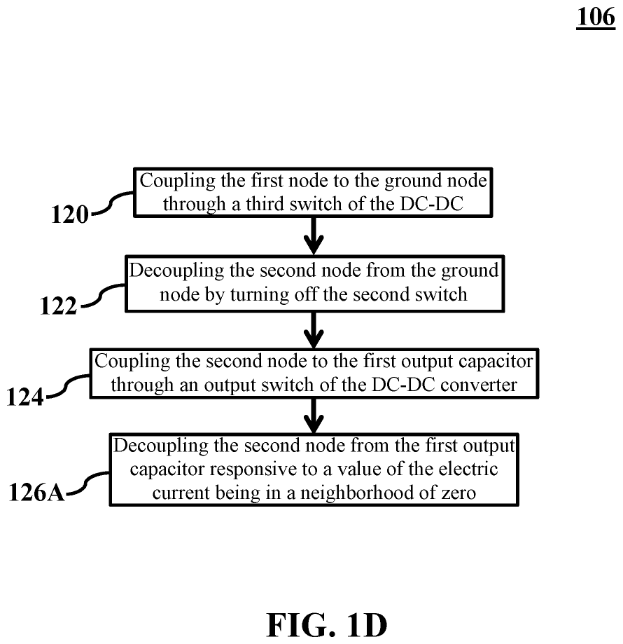

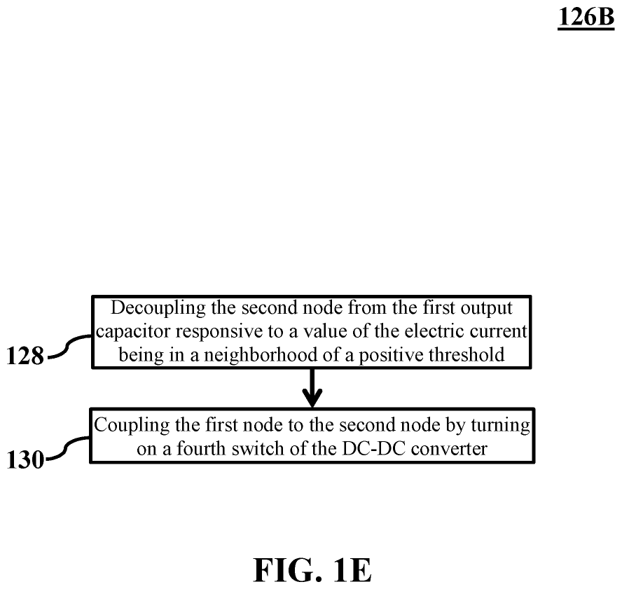

FIG. 1A shows a flowchart of a method for DC-DC conversion, consistent with one or more exemplary embodiments of the present disclosure. In an exemplary embodiment, a method 100 may include converting an input voltage to a set of output voltages by selecting an output capacitor in a set of output capacitors of a DC-DC converter (step 102), charging an inductor of the DC-DC converter (step 104), and discharging an electric current passing through the inductor into the output capacitor (step 106). In an exemplary embodiment, different steps of method 100 may be implemented utilizing a processor, facilitating a fully digital, programmable, and reconfigurable implementation of method 100. An exemplary fully digital implementation of method 100 may decrease a sensitivity of voltage conversion to process, voltage, and temperature (PVT) variations.

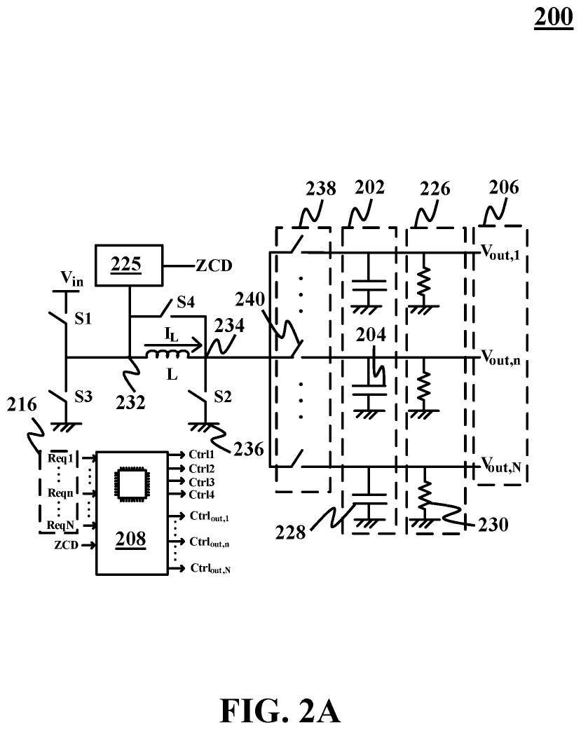

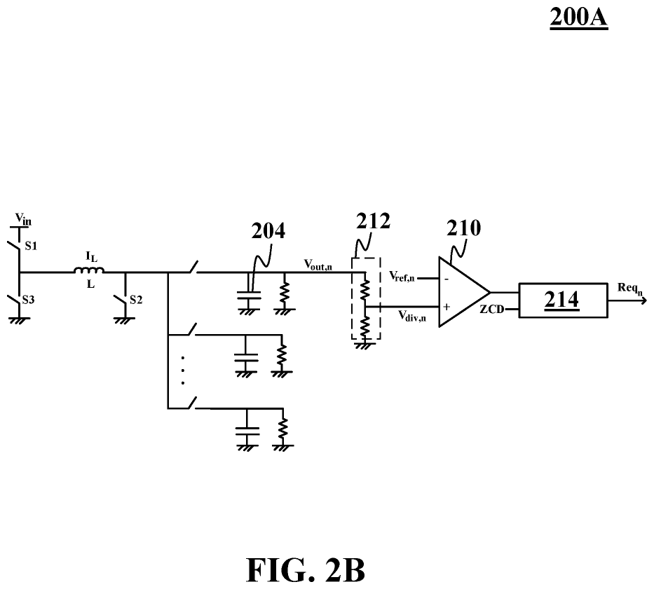

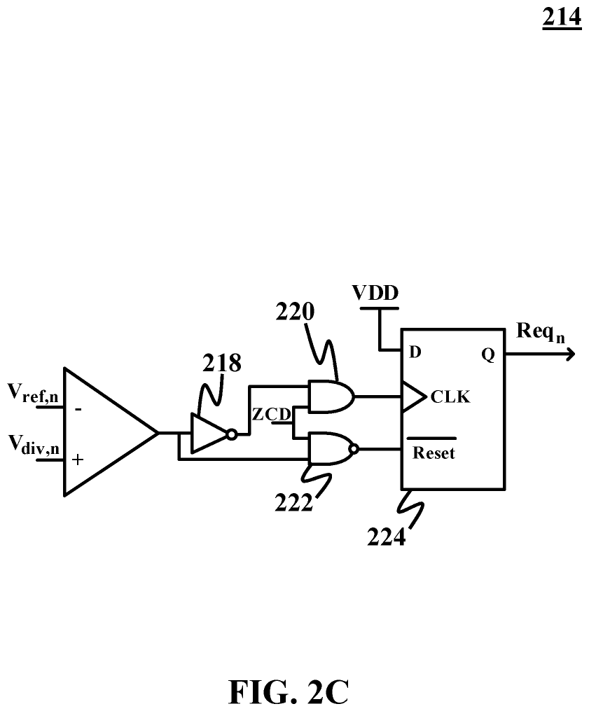

FIG. 2A shows a schematic of a DC-DC converter, consistent with one or more exemplary embodiments of the present disclosure. Referring to FIGS. 1A and 2A, in an exemplary embodiment, different steps of method 100 may be implemented utilizing a DC-DC converter 200. In an exemplary embodiment, DC-DC converter 200 may include a set of output capacitors 202 and an inductor L. In an exemplary embodiment, set of output capacitors 202 may include an output capacitor 204. In an exemplary embodiment, DC-DC converter may convert an input voltage Vᵢₙ to a set of output voltages 206. In an exemplary embodiment, method 100 may include a switching method for converting input voltage Vᵢₙ to set of output voltages 206. In an exemplary embodiment, DC-DC converter may include a processor 208. In an exemplary embodiment, processor 208 may turn on and off switches in DC-DC converter 200 by generating a respective control signal for each switch.

For further detail with respect to step 102, FIG. 1B shows a flowchart of a method for selecting an output capacitor in a set of output capacitors, consistent with one or more exemplary embodiments of the present disclosure. Referring to FIGS. 1B and 2A, in an exemplary embodiment, output capacitor 204 may be selected utilizing processor 208. In an exemplary embodiment, selecting output capacitor 204 may include generating a set of request signals (step 108) and selecting a first request signal in the set of request signals by prioritizing the set of request signals (step 110).

FIG. 2B shows a schematic of a DC-DC converter including a voltage divider and a comparator, consistent with one or more exemplary embodiments of the present disclosure. In an exemplary embodiment, DC-DC converter 200A may include a first implementation of DC-DC converter 200. In an exemplary embodiment, DC-DC converter 200A may include a comparator 210, a voltage divider 212, and a digital circuit 214. In an exemplary embodiment, output capacitor 204 may be selected responsive to an output voltage V_{out,n} of output capacitor 204 being less than a reference voltage V_{ref,n} in a set of reference voltages. In an exemplary embodiment, output voltage V_{out,n} may be compared with reference voltage V_{ref,n} utilizing comparator 210. In an exemplary embodiment, output voltage V_{out,n} may not be in an input voltage range of comparator 210. Therefore, in an exemplary embodiment, reference voltage V_{ref,n} may be compared with output voltage V_{out,n} through voltage divider 212. As a result, reference voltage V_{ref,n} may be compared with a voltage V_{div,n} instead of output voltage V_{out,n}. In an exemplary embodiment, voltage V_{div,n} may be in the input voltage range of comparator 210.

Referring to FIGS. 1B and 2A, in an exemplary embodiment, step 108 may include generating a set of request signals. In an exemplary embodiment, generating a set of request signals 216 may include generating each request signal in set of request signals 216 responsive to a voltage level of a respective output capacitor in set of output capacitors 202 being less than a respective reference voltage in the set of reference voltages. In an exemplary embodiment, request signal Reqₙ may be associated with output capacitor 204. Specifically, in an exemplary embodiment, request signal Reqₙ may be generated from output voltage V_{out,n} of output capacitor 204. In an exemplary embodiment, request signal Reqₙ may be generated utilizing digital circuit 214. An exemplary output of comparator 210 may be fed to digital circuit 214. Therefore, in an exemplary embodiment, digital circuit 214 may generate request signal Reqₙ responsive to voltage V_{div,n} being less than reference voltage V_{ref,n}.

FIG. 2C shows a schematic of a digital circuit, consistent with one or more exemplary embodiments of the present disclosure. In an exemplary embodiment, digital circuit 214 may include a NOT gate 218, an AND gate 220, a NAND gate 222, and a D flip-flop 224. Referring to FIGS. 2A and 2C, in an exemplary embodiment, DC-DC converter 200 may further include a zero-current detector 225. In an exemplary embodiment, zero-current detector 225 may detect a change in a level of an electric current I_{L} passing through inductor L from positive to negative. In an exemplary embodiment, zero-current detector 225 may generate a signal ZCD responsive to electric current I_{L} reaching zero. In an exemplary embodiment, when voltage V_{div,n} is larger than reference voltage V_{ref,n}, an output of comparator 210 may include a logical 1 value and signal ZCD may include a logical 1 value. As a result, a reset signal of D flip-flop 224 may include a logical 0 value and request signal Reqₙ may include a logical 0 value. In contrast, an exemplary output of comparator 210 may include a logical 0 value when voltage V_{div,n} is less than reference voltage V_{ref,n}. Thus, in an exemplary embodiment, reset signal may include a logical 1 value and request signal Reqₙ may still include a logical 0 value. Therefore, in an exemplary embodiment, a clock signal CLK of D flip-flop 224 may become a logical 1 value, changing a value of request signal Reqₙ from a logical 0 value to a logical 1 value. In other words, in an exemplary embodiment, request signal Reqₙ may be generated responsive to voltage V_{div,n} being less than reference voltage V_{ref,n}.

Referring to FIGS. 1B and 2A, in an exemplary embodiment, step 110, may include selecting request signal Reqₙ. In an exemplary embodiment, request signal Reqₙ may be selected by prioritizing set of request signals 216. Two or more exemplary output capacitors in set of output capacitors 202 may require to be charged simultaneously to maintain corresponding output voltages. However, only one exemplary output capacitor may be charged at any given instant. Therefore, exemplary request signals may be prioritized and an output capacitor with highest priority may be selected for charging. In an exemplary embodiment, output capacitor 204 may be of highest priority among output capacitors that require to be charged. An exemplary priority measure may include a priority in generation of request signals. In other words, an exemplary request signal that is generated prior to other request signals may be selected as a highest priority request signal. Besides, in an exemplary embodiment, arbitrary priority levels may be assigned to different request signals set of request signals 216.

In an exemplary embodiment, processor 208 may generate a set of enabling signals from set of request signals 216. Each exemplary enabling signal may be utilized for charging a respective output capacitor in set of output capacitors 202. In an exemplary embodiment, processor 208 may prioritize set of request signals 216 by activing only one enabling signal corresponding to output capacitor 204. Activating an exemplary enabling signal may be referred to as assigning a logical 1 value to an enabling signal. A logical 0 value may be assigned to all other exemplary enabling signals.

Referring to FIGS. 1A, 1B, and 2A, in an exemplary embodiment, selecting output capacitor 204 in step 102 may further include setting set of request signals 216 to an empty set (step 112). An exemplary voltage source may provide input voltage Vᵢₙ to DC-DC converter 200. In an exemplary embodiment, an average of electric current I_{L} drawn from the voltage source may increase by increasing a consumed power of a set of loads 226 connected to DC-DC converter 200. An exemplary excessive current may be drawn from the voltage source when set of loads 226 consume excessive power, resulting in possible damage to the voltage source. In an exemplary embodiment, setting set of request signals 216 to the empty set may prevent set of loads 226 to draw an excessive current from the voltage source because no output capacitor may be charged when no request signal is generated.

In an exemplary embodiment, set of request signals 216 may be set to an empty set responsive to a consumed energy of a voltage source being higher than an input energy threshold. To detect an excessive current drawn from an exemplary voltage source, a consumed energy of the voltage source in a specified time interval may be calculated. In an exemplary embodiment, consumed energy of the voltage source is calculated by calculating a transferred energy from inductor L to each output capacitor in set of output capacitors 202 in the specified time interval. A transferred energy from an exemplary voltage source to set of output capacitors 202 may be calculated by a Coulomb counting method, as described below.

In an exemplary embodiment, transferred energy from inductor L to each output capacitor may depend on an operating mode of DC-DC converter 200, that is, a boost mode or a buck mode. In an exemplary embodiment, DC-DC converter 200 may operate in a boost mode when V_{out,n}>Vᵢₙ. In an exemplary embodiment, DC-DC converter 200 may operate in a buck mode when Vₒᵤₜₙ <Vᵢₙ. In an exemplary embodiment, when inductor L is charging, electric current I_{L} may be equal to

$${I}_{L} = \frac{1}{L} {V}_{in} \cdot t ,$$

where t∈(0, T_{Charge,n}) and T_{charge,n} is a duration of charging inductor L. In an exemplary embodiment, transferred energy from inductor L to output capacitor 204 in a buck mode may be calculated according to an operation defined by the following:

$${E}_{Buck , n} = {\int }_{0}^{{T}_{Charge , n}} {V}_{i n} \cdot {I}_{L} d t = \frac{{V}_{i n}^{2} \cdot {T}_{Charge , n}^{2}}{2 L} , \qquad Equation ( 1 )$$

In an exemplary embodiment, when DC-DC converter operates in the boost mode and inductor L is discharging into output capacitor 204, electric current I_{L} may be calculated according to an operation defined by the following:

$${I}_{L} = - \frac{1}{L} ( {V}_{out , i} - {V}_{i n} ) \cdot ( t - {T}_{Charge , n} ) + \frac{1}{L} {V}_{i n} \cdot {T}_{Charge , n} , \qquad Equation ( 2 )$$

where t∈(T_{Charge,n}, T_{Charge,n}+T_{Discharge,n}) and T_{Discharge,n} is a duration of discharging inductor L into output capacitor 204. In an exemplary embodiment, transferred energy from inductor L to output capacitor 204 in the boost mode may be calculated according to an operation defined by the following:

$${E}_{B o ost , n} = {\int }_{0}^{{T}_{Charge , n}} {V}_{i n} \cdot {I}_{L} d t + {\int }_{{T}_{Charge , n}}^{{T}_{Charge , n} + {T}_{Discharge , n}} {V}_{i n} \cdot {I}_{L} dt = \frac{{V}_{i n}^{2} \cdot {T}_{C h a rge , n}^{2}}{2 L} + \frac{{V}_{i n}^{3} \cdot {T}_{C h a rge , n}^{2}}{2 L \cdot ( {V}_{out , n} - {V}_{i n} )} \qquad Equation ( 3 )$$

To identify whether an excessive current is drawn from an exemplary voltage source, transferred energy in a specified time interval may be calculated. In an exemplary embodiment, a total transferred energy Eₜₒₜₐₗ in a specified time interval may be equal to Eₜₒₜₐₗ=Σₘ₌₁^{M}qₘE_{Buck,m}+(1−qₘ), where qₘ=1 for the buck mode, q₌₀ in the boost mode, and M is a total number of output capacitors that are charged in a specified time interval. In an exemplary embodiment, total transferred energy Eₜₒₜₐₗ may be compared with an input energy threshold E_{th,in}. In an exemplary embodiment, input energy threshold E_{th,in} may be obtained from a maximum current that a voltage source can provide to set of output capacitors 202. In an exemplary embodiment, when Eₜₒₜₐₗ>E_{th,in}, an excessive current may be drawn from the voltage source and set of request signals 216 may be set to an empty set to protect the voltage source from excessive current until a condition Eₜₒₜₐₗ<E_{th,in} is satisfied. An exemplary state of charge of the voltage source may be obtained from total transferred energy Eₜₒₜₐₗ and by measuring voltage input Vᵢₙ in different time instances.

In an exemplary embodiment, selecting output capacitor 204 in step 102 may further include eliminating an overcurrent request signal from set of request signals 216 (step 114). An exemplary overcurrent request signal may be eliminated responsive to a transferred energy from the voltage source to an overcurrent output capacitor 228 in set of output capacitors 202 being higher than an output energy threshold. In an exemplary embodiment, a load 230 in set of loads 230 may be connected to overcurrent output capacitor 228. In an exemplary embodiment, load 230 may consume excessive energy that may pertain to a damage in load 230. In an exemplary embodiment, DC-DC converter 200 may protect load 230 from being overcurrent. An exemplary excessive current may be drawn from overcurrent output capacitor 228. In an exemplary embodiment, similar to transferred energy calculation in step 112, a transferred energy from the voltage source to overcurrent output capacitor 228 may be calculated. An exemplary voltage level of overcurrent output capacitor 228 may be smaller than a reference voltage when an overcurrent occurs in load 230. As a result, an exemplary overcurrent request signal in set of request signals 216 may be generated. In an exemplary embodiment, transferred energy from the voltage source to output capacitor 228 may be calculated according to one of Equation (1) or Equation (2). Therefore, an exemplary overcurrent request signal may be eliminated from set of request signals 216 when E_{Boost}>E_{th,out} or E_{Buck}>E_{th,out}, depending on a buck or a boost operating mode of DC-DC converter for output capacitor 228.

In an exemplary embodiment, when DC-DC converter 200 starts working, set of output voltages 206 may be zero. As a result, in an exemplary embodiment, set of output capacitors 202 may require to continuously be charged until each output voltage reaches a respective reference voltage, resulting in a steep increase in output voltages. In an exemplary embodiment, to avoid a steep increase in output voltages, a transferred energy from the voltage source to each output capacitor may be found by coulomb counting in step 114, and charging of overcurrent outputs may be prevented.

In an exemplary embodiment, step 104 may include charging inductor L by applying input voltage Vᵢₙ to inductor L. In further detail with respect to step 104, FIG. 1C shows a flowchart of a method for applying an input voltage to an inductor, consistent with one or more exemplary embodiments of the present disclosure. Referring to FIGS. 1C and 2A, in an exemplary embodiment, applying input voltage Vᵢₙ to inductor L may include applying input voltage Vᵢₙ to a first node 232 of inductor L (step 116) and coupling a second node 234 of inductor L to a ground node 236 (step 118).

For further detail regarding step 116, in an exemplary embodiment, input voltage Vᵢₙ may be applied to first node 232 through a first switch S1 of DC-DC converter 200. In an exemplary embodiment, processor 208 may generate a control signal Ctrl1 for charging inductor L. In an exemplary embodiment, control signal Ctrl1 may be applied to a gate of switch S1 through a driver circuit of DC-DC converter 200. As a result, in an exemplary embodiment, switch S1 may be turned on and input voltage Vᵢₙ may be applied to first node 232.

In further detail with regard to step 118, in an exemplary embodiment, second node 234 may be coupled to ground node 236 through a second switch S2 of DC-DC converter 200. In an exemplary embodiment, processor 208 may generate a control signal Ctrl2 for charging inductor L. In an exemplary embodiment, control signal Ctrl2 may be applied to a gate of switch S2 through a driver circuit of DC-DC converter 200. As a result, in an exemplary embodiment, switch S2 may be turned on and second node 234 may be coupled to ground node 236.

In an exemplary embodiment, a load connected to output capacitor 204 may be short-circuited. As a result, in an exemplary embodiment, output capacitor 204 may require to be charged continuously. In an exemplary embodiment, to avoid feeding excessive current to a short-circuited load, a duration of charging inductor L may be limited and may be equal to T_{charge,n}. As a result, in an exemplary embodiment, stored energy in inductor L may be limited in each time of charging and an excessive current may not be drawn by a short-circuited load connected to output capacitor 204. Therefore, in an exemplary embodiment, DC-DC converter 200 may be protected from short-circuited loads.

Referring again to FIGS. 1A and 2A, in an exemplary embodiment, step 106 may include discharging electric current I_{L} into output capacitor 204. For further detail regarding step 106, FIG. 1D shows a flowchart of a method for applying an input voltage to an inductor, consistent with one or more exemplary embodiments of the present disclosure. Referring to FIGS. 1D and 2A, in an exemplary embodiment, discharging electric current I_{L} may include coupling first node 232 to ground node 236 (step 120), decoupling second node 234 from ground node 236 (step 122), and coupling second node 234 to output capacitor 204 (step 124). In an exemplary embodiment, DC-DC converter 200 may further include a set of output switches 238. Each exemplary output switch in set of output switches 238 may be connected between second node 234 and a respective output capacitor in set of output capacitors 202. Specifically, in an exemplary embodiment, an output switch 240 in set of output switches 238 may be connected between second node 234 and output capacitor 204. After charging inductor L, in an exemplary embodiment, processor 208 may activate an enabling signal for output capacitor 204. Then, in an exemplary embodiment, processor 208 may accordingly generate a set of control signals to generate a path between inductor L and output capacitor 204. Each exemplary control signal may be applied to a gate of a respective switch of DC-DC converter 200 to discharge electric current I_{L} into output capacitor 204.

In further detail with respect to step 120, in an exemplary embodiment, first node 232 may be coupled to ground node 236 through a third switch S3 of DC-DC converter 200. In an exemplary embodiment, first node 232 may be coupled to ground node 236 responsive to DC-DC converter 200 operating in a buck mode. Meanwhile, in an exemplary embodiment, first node 232 may be decoupled from input voltage Vᵢₙ by turning off switch S1 responsive to DC-DC converter 200 operating in a buck mode. In an exemplary embodiment, processor 208 may generate a control signal Ctrl3 for discharging inductor L. In an exemplary embodiment, control signal Ctrl3 may be applied to a gate of switch S3 through a driver circuit of DC-DC converter 200. As a result, in an exemplary embodiment, switch S3 may be turned on and first node 232 may be coupled to ground node 236. In contrast, in an exemplary embodiment, switch S1 may be remained turned on and switch S3 may be remained turned off responsive to DC-DC converter 200 operating in a boost mode.

For further detail regarding step 122, in an exemplary embodiment, second node 234 may be decoupled from ground node 236 by turning off switch S2. In an exemplary embodiment, processor 208 may turn off switch S2 by deactivating control signal Ctrl2.

In further detail with regard to step 124, in an exemplary embodiment, second node 234 may be coupled to output capacitor 204 through output switch 240. In an exemplary embodiment, processor 208 may generate a control signal Ctrl_{out,n} to couple second node 234 to output capacitor 204. In an exemplary embodiment, control signal Ctrl_{out,n} may be applied to a gate of output switch 240 through a driver circuit of DC-DC converter 200. As a result, in an exemplary embodiment, output switch 240 may be turned on and second node 234 may be coupled to output capacitor 204.

Referring to FIGS. 1A, 1D, and 2A, in an exemplary embodiment, discharging electric current I_{L} in step 106 may further include decoupling second node 234 from output capacitor 204 (step 126A). In an exemplary embodiment, step 126A may include a first implementation of decoupling second node 234 from output capacitor 204. In an exemplary embodiment, second node 234 may be decoupled from output capacitor 204 by turning off output switch 240. In an exemplary embodiment, output switch 240 may be turned off responsive to a value of electric current I_{L} being in a neighborhood of zero. In an exemplary embodiment, the neighborhood of zero may be calculated according to an operation defined by the following:

$$\vert {I}_{L} \vert < \frac{{V}_{i n} {T}_{charge , n}}{L} \sqrt{ε} \qquad Inequality ( 1 )$$

where ε is a reduction in an efficiency of DC-DC converter 200 due to an imprecise current detection of zero-current detector 225. In an exemplary embodiment, for one percent reduction in an efficiency of DC-DC converter 200, ε may be equal to 0.01.

In an exemplary embodiment, electric current I_{L} may flow from the voltage source to output capacitor 204 when electric current I_{L} is positive. Therefore, in an exemplary embodiment, output capacitor 204 may be charged to output voltage V_{out,n}. In contrast, in an exemplary embodiment, electric current I_{L} may flow from output capacitor 204 to the voltage source when electric current I_{L} is negative. Therefore, in an exemplary embodiment, output capacitor 204 may be discharged. To avoid discharging output capacitor 204, in an exemplary embodiment, output switch 240 may be turned off when electric current I_{L} crosses zero. In an exemplary embodiment, zero-current detector 225 may generate a signal ZCD and responsive to electric current I_{L} crossing zero. Then in an exemplary embodiment, zero-current detector 225 may send signal ZCD to processor 208. In an exemplary embodiment, processor 208 may then deactivate control signal Ctrl_{out,n}. As a result, in an exemplary embodiment, output switch 240 may be turned off.

FIG. 1E shows a flowchart of a method for discharging an electric current into an output capacitor, consistent with one or more exemplary embodiments of the present disclosure. Referring to FIGS. 1A, 1E, and 2A, in an exemplary embodiment, discharging electric current I_{L} in step 106 may further include decoupling second node 234 from output capacitor 204 (method 126B). In an exemplary embodiment, method 126B may include a second implementation of decoupling second node 234 from output capacitor 204. In an exemplary embodiment, discharging electric current I_{L} may further include decoupling second node 234 from output capacitor 204 (step 128) and coupling first node 232 to second node 234 (step 130).

For further detail with respect to step 128, in an exemplary embodiment, second node 234 may be decoupled from output capacitor 204 by turning off output switch 240. In an exemplary embodiment, output switch 240 may be turned off responsive to a value of electric current I_{L} being in a neighborhood of a positive threshold Iₜₕ. As a result, in an exemplary embodiment, charging output capacitor 204 may be stopped before fully discharging electric current I_{L}. In an exemplary embodiment, the neighborhood of positive threshold Iₜₕ may be calculated according to an operation defined by the following:

$$\vert {I}_{L} \vert < \vert {I}_{t h} - \frac{1}{L} \sqrt{\vert {I}_{t h}^{2} {L}^{2} - L ε ( 2 {I}_{t h} + \frac{{V}_{i n} {T}_{charge , n}}{L} ) \times {V}_{i n} {T}_{Charge , n} \vert} \vert \qquad Inequality ( 2 )$$

where ε is a reduction in an efficiency of DC-DC converter 200 due to an imprecise current detection of a current-detection circuit.

In further detail regarding step 130, in an exemplary embodiment, coupling first node 232 to second node 234 may include turning on fourth switch S4 of DC-DC converter 200. In an exemplary embodiment, fourth switch S4 may be connected in parallel with inductor L. In an exemplary embodiment, coupling first node 232 to second node 234 before fully discharging inductor L may preserve electric current I_{L} flowing in a loop between first node 232 and second node 234.

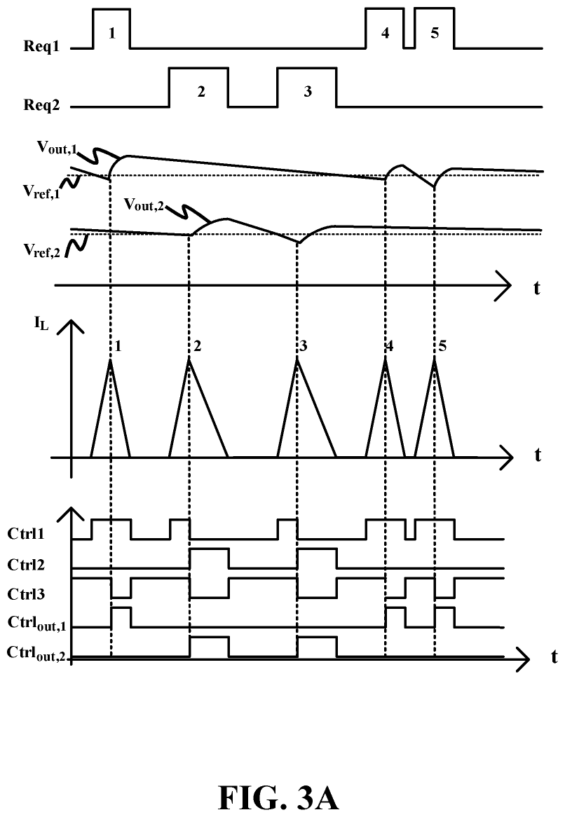

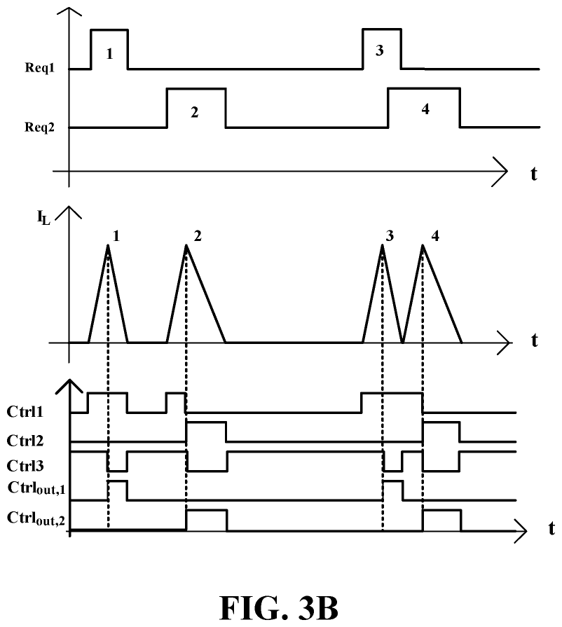

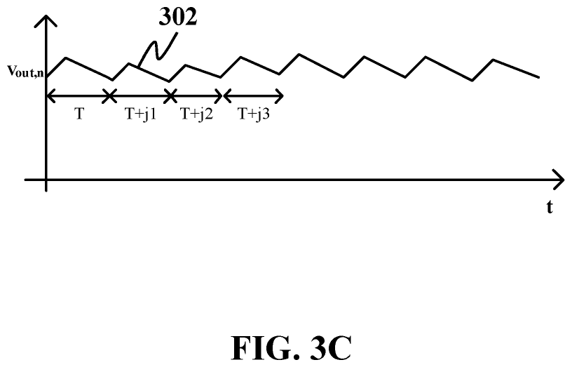

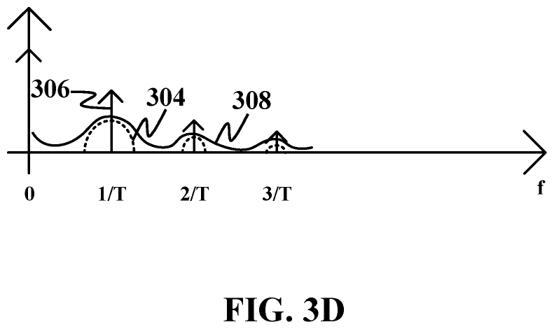

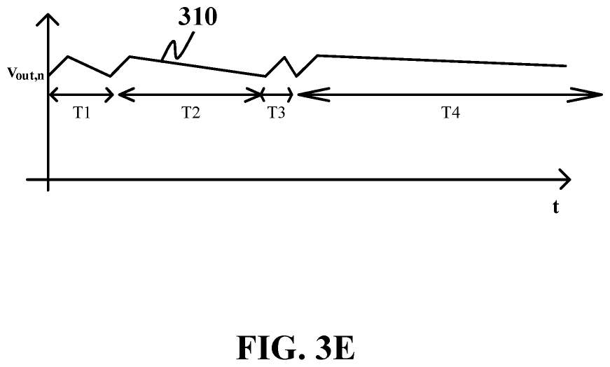

FIG. 3A shows control signals, an electric current, and output voltages for non-overlapped request signals, consistent with one or more exemplary embodiments of the present disclosure. Referring to FIGS. 2A, 2B, and 3A, in an exemplary embodiment, an output voltage V_{out,1} may become smaller than a reference voltage V_{ref,1} due to a power consumption of a load connected to an output capacitor of DC-DC converter 200. Thus, in an exemplary embodiment, digital circuit 214 may generate a request signal Req₁ and may preserve a value of request signal Req₁ during a time interval 1. In an exemplary embodiment, since only request signal Req₁ is generated in time interval 1, set of request signals 216 may include only request signal Req₁. Therefore, in an exemplary embodiment, processor 208 may select request signal Req₁ as a highest priority request signal. In an exemplary embodiment, processor 208 may activate control signal Ctrl1 and control signal Ctrl3 to charge inductor L. In an exemplary embodiment, electric current I_{L} may increase to a peak value in time interval 1. Then, in an exemplary embodiment, processor 208 may deactivate control signal Ctrl3 to decouple second node 234 from ground node 236. In an exemplary embodiment, DC-DC converter 200 may operate in a boost mode for output voltage V_{out,1}, that is, V_{out,1}>Vᵢₙ. As a result, in an exemplary embodiment, control signal Ctrl1 may remain activated when inductor L is discharging. Meanwhile, in an exemplary embodiment, processor 208 may activate a control signal Ctrl_{out,1} to turn on a first output switch in set of output switches 238, making a path for discharging inductor L. By discharging inductor L, in an exemplary embodiment, output voltage V_{out,1} become larger than reference voltage V_{ref,1}. In an exemplary embodiment, electric current I_{L} may be decreasing when control signal Ctrl_{out,1} is activated. In an exemplary embodiment, when electric current I_{L} reaches zero, zero-current detector 225 may generate signal ZCD. As a result, in an exemplary embodiment, digital circuit 214 may deactivate control signal Req₁ and processor 208 may deactivate control signal Ctrl1.

In an exemplary embodiment, a similar procedure may be performed in a time interval 2 to charge an output capacitor of a second output of DC-DC converter 200. In an exemplary embodiment, DC-DC converter 200 may operate in a buck mode for an output voltage V_{out,2}. Therefore, in an exemplary embodiment, processor 208 may deactivate control signal Ctrl1 and activate a control signal Ctrl2 for discharging inductor L.

FIG. 3B shows control signals, an electric current, and output voltages for overlapped request signals, consistent with one or more exemplary embodiments of the present disclosure. Referring to FIGS. 2A and 3B, in an exemplary embodiment, a request signal Req₁ may be generated in a time interval 3 and inductor L may be charged accordingly. Meanwhile, in an exemplary embodiment, a request signal Req2 may be generated in a time interval 4 that overlaps with time interval 3. As a result, in an exemplary embodiment, set of request signals 216 may include request signal Req₁ and request signal Req2. In an exemplary embodiment, request signal Req₁ may be selected because request signal Req₁ is requested prior to request signal Req2. In an exemplary embodiment, after charging an output capacitor of request signal Req₁, inductor L may be charged and discharged to charge an output capacitor of request signal Req2.

FIG. 3C shows an output voltage provided to a constant load, consistent with one or more exemplary embodiments of the present disclosure. Referring to FIGS. 2A and 3C, in an exemplary embodiment, a voltage 302 of output voltage Vₒᵤₜₙ may be provided to a constant load, that is, a load that draws a constant current in time. In an exemplary embodiment, since output capacitor 204 may not be periodically charged, an overall charging and discharging time of output capacitor 204 may include a jitter in different charging and discharging turns. Specifically, in an exemplary embodiment, charging and discharging of output capacitor 204 may take T, T+j₁, T+j₂, and T+j₃ seconds in four consecutive charging turns, where T is a period of charging set of output capacitors 202 and j₁, j₂, and j₃ are jitters of charging and discharging output capacitor 204.

FIG. 3D shows a spectrum of an output voltage, consistent with one or more exemplary embodiments of the present disclosure. Referring to FIGS. 1A, 3C, and 3D, in an exemplary embodiment, a spectrum of voltage 302 may include a spectrum 304. In an exemplary embodiment, a spectrum 306 may include a spectrum of an output voltage that charges with fixed frequency. As a result, in an exemplary embodiment, spectrum 306 may include a number of harmonics at r/T Hz, where r>1 is a number of harmonics. In contrast to spectrum 306, in an exemplary embodiment, spectrum 304 may be flattened around each harmonic. Therefore, in an exemplary embodiment, method 100 may be more robust to noise than fixed-frequency voltage conversion methods such as pulse width modulation methods.

FIG. 3E shows an output voltage provided to a variant load, consistent with one or more exemplary embodiments of the present disclosure. Referring to FIGS. 1A, 3D, and 3E, in an exemplary embodiment, a spectrum 308 may include a spectrum of an output voltage 310 provided to a variant load. In an exemplary embodiment, a charging and discharging times of an output voltage 310 may be independent in different turns for a variant load, that is, a load that draws a variant current from an output capacitor in time. As a result, in an exemplary embodiment, spectrum 308 may be spread in different frequencies rather than being concentrated around different harmonics. As a result, in an exemplary embodiment, a noise immunity of method 100 may enhance for variant loads.

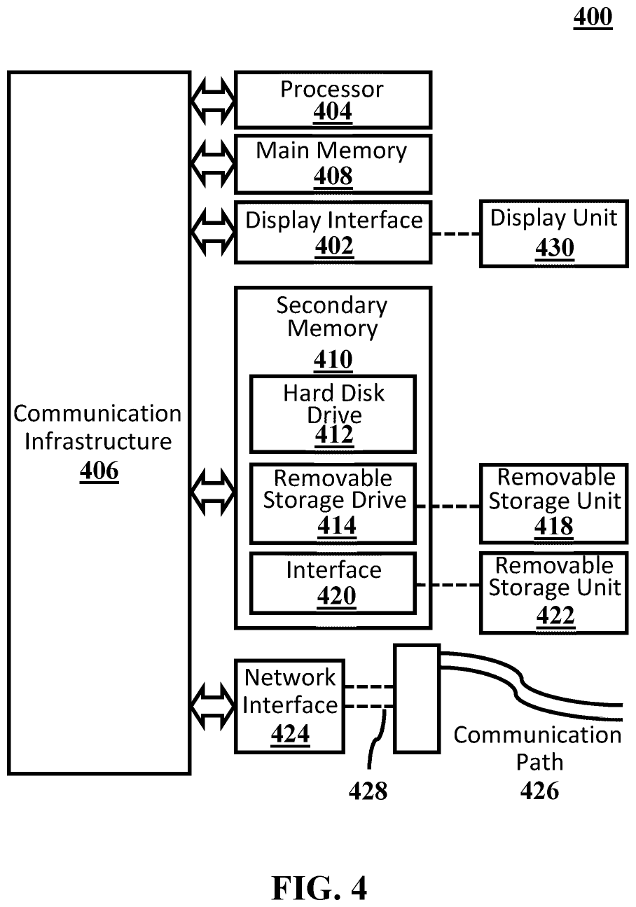

FIG. 4 shows an example computer system 400 in which an embodiment of the present invention, or portions thereof, may be implemented as computer-readable code, consistent with exemplary embodiments of the present disclosure. For example, different steps of method 100 may be implemented in computer system 400 using hardware, software, firmware, tangible computer readable media having instructions stored thereon, or a combination thereof and may be implemented in one or more computer systems or other processing systems. Hardware, software, or any combination of such may embody any of the modules and components in FIGS. 1A-2C.

If programmable logic is used, such logic may execute on a commercially available processing platform or a special purpose device. One ordinary skill in the art may appreciate that an embodiment of the disclosed subject matter can be practiced with various computer system configurations, including multi-core multiprocessor systems, minicomputers, mainframe computers, computers linked or clustered with distributed functions, as well as pervasive or miniature computers that may be embedded into virtually any device.

For instance, a computing device having at least one processor device and a memory may be used to implement the above-described embodiments. A processor device may be a single processor, a plurality of processors, or combinations thereof. Processor devices may have one or more processor “cores.”

An embodiment of the invention is described in terms of this example computer system 400. After reading this description, it will become apparent to a person skilled in the relevant art how to implement the invention using other computer systems and/or computer architectures. Although operations may be described as a sequential process, some of the operations may in fact be performed in parallel, concurrently, and/or in a distributed environment, and with program code stored locally or remotely for access by single or multi-processor machines. In addition, in some embodiments the order of operations may be rearranged without departing from the spirit of the disclosed subject matter.

Processor device 404 may be a special purpose (e.g., a graphical processing unit) or a general-purpose processor device. As will be appreciated by persons skilled in the relevant art, processor device 404 may also be a single processor in a multi-core/multiprocessor system, such system operating alone, or in a cluster of computing devices operating in a cluster or server farm. Processor device 404 may be connected to a communication infrastructure 406, for example, a bus, message queue, network, or multi-core message-passing scheme.

In an exemplary embodiment, computer system 400 may include a display interface 402, for example a video connector, to transfer data to a display unit 430, for example, a monitor. Computer system 400 may also include a main memory 408, for example, random access memory (RAM), and may also include a secondary memory 410. Secondary memory 410 may include, for example, a hard disk drive 412, and a removable storage drive 414. Removable storage drive 414 may include a floppy disk drive, a magnetic tape drive, an optical disk drive, a flash memory, or the like. Removable storage drive 414 may read from and/or write to a removable storage unit 418 in a well-known manner. Removable storage unit 418 may include a floppy disk, a magnetic tape, an optical disk, etc., which may be read by and written to by removable storage drive 414. As will be appreciated by persons skilled in the relevant art, removable storage unit 418 may include a computer usable storage medium having stored therein computer software and/or data.

In alternative implementations, secondary memory 410 may include other similar means for allowing computer programs or other instructions to be loaded into computer system 400. Such means may include, for example, a removable storage unit 422 and an interface 420. Examples of such means may include a program cartridge and cartridge interface (such as that found in video game devices), a removable memory chip (such as an EPROM, or PROM) and associated socket, and other removable storage units 422 and interfaces 420 which allow software and data to be transferred from removable storage unit 422 to computer system 400.

Computer system 400 may also include a communications interface 424. Communications interface 424 allows software and data to be transferred between computer system 400 and external devices. Communications interface 424 may include a modem, a network interface (such as an Ethernet card), a communications port, a PCMCIA slot and card, or the like. Software and data transferred via communications interface 424 may be in the form of signals, which may be electronic, electromagnetic, optical, or other signals capable of being received by communications interface 424. These signals may be provided to communications interface 424 via a communications path 426. Communications path 426 carries signals and may be implemented using wire or cable, fiber optics, a phone line, a cellular phone link, an RF link or other communications channels.

In this document, the terms “computer program medium” and “computer usable medium” are used to generally refer to media such as removable storage unit 418, removable storage unit 422, and a hard disk installed in hard disk drive 412. Computer program medium and computer usable medium may also refer to memories, such as main memory 408 and secondary memory 410, which may be memory semiconductors (e.g. DRAMs, etc.).

Computer programs (also called computer control logic) are stored in main memory 408 and/or secondary memory 410. Computer programs may also be received via communications interface 424. Such computer programs, when executed, enable computer system 400 to implement different embodiments of the present disclosure as discussed herein. In particular, the computer programs, when executed, enable processor device 404 to implement the processes of the present disclosure, such as the operations in method 100 illustrated by flowcharts of FIGS. 1A-1E discussed above. Accordingly, such computer programs represent controllers of computer system 400. Where an exemplary embodiment of method 100 is implemented using software, the software may be stored in a computer program product and loaded into computer system 400 using removable storage drive 414, interface 420, and hard disk drive 412, or communications interface 424.

Embodiments of the present disclosure also may be directed to computer program products including software stored on any computer useable medium. Such software, when executed in one or more data processing device, causes a data processing device to operate as described herein. An embodiment of the present disclosure may employ any computer useable or readable medium. Examples of computer useable mediums include, but are not limited to, primary storage devices (e.g., any type of random access memory), secondary storage devices (e.g., hard drives, floppy disks, CD ROMS, ZIP disks, tapes, magnetic storage devices, and optical storage devices, MEMS, nanotechnological storage device, etc.).

The embodiments have been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

While the foregoing has described what may be considered to be the best mode and/or other examples, it is understood that various modifications may be made therein and that the subject matter disclosed herein may be implemented in various forms and examples, and that the teachings may be applied in numerous applications, only some of which have been described herein. It is intended by the following claims to claim any and all applications, modifications and variations that fall within the true scope of the present teachings.

Unless otherwise stated, all measurements, values, ratings, positions, magnitudes, sizes, and other specifications that are set forth in this specification, including in the claims that follow, are approximate, not exact. They are intended to have a reasonable range that is consistent with the functions to which they relate and with what is customary in the art to which they pertain.

The scope of protection is limited solely by the claims that now follow. That scope is intended and should be interpreted to be as broad as is consistent with the ordinary meaning of the language that is used in the claims when interpreted in light of this specification and the prosecution history that follows and to encompass all structural and functional equivalents. Notwithstanding, none of the claims are intended to embrace subject matter that fails to satisfy the requirement of Sections 101, 102, or 103 of the Patent Act, nor should they be interpreted in such a way. Any unintended embracement of such subject matter is hereby disclaimed.

Except as stated immediately above, nothing that has been stated or illustrated is intended or should be interpreted to cause a dedication of any component, step, feature, object, benefit, advantage, or equivalent to the public, regardless of whether it is or is not recited in the claims.

It will be understood that the terms and expressions used herein have the ordinary meaning as is accorded to such terms and expressions with respect to their corresponding respective areas of inquiry and study except where specific meanings have otherwise been set forth herein. Relational terms such as first and second and the like may be used solely to distinguish one entity or action from another without necessarily requiring or implying any actual such relationship or order between such entities or actions. The terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element proceeded by “a” or “an” does not, without further constraints, preclude the existence of additional identical elements in the process, method, article, or apparatus that comprises the element.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various implementations. This is for purposes of streamlining the disclosure, and is not to be interpreted as reflecting an intention that the claimed implementations require more features than are expressly recited in each claim. Rather, as the following claims reflect, inventive subject matter lies in less than all features of a single disclosed implementation. Thus, the following claims are hereby incorporated into the Detailed Description, with each claim standing on its own as a separately claimed subject matter.

While various implementations have been described, the description is intended to be exemplary, rather than limiting and it will be apparent to those of ordinary skill in the art that many more implementations and implementations are possible that are within the scope of the implementations. Although many possible combinations of features are shown in the accompanying figures and discussed in this detailed description, many other combinations of the disclosed features are possible. Any feature of any implementation may be used in combination with or substituted for any other feature or element in any other implementation unless specifically restricted. Therefore, it will be understood that any of the features shown and/or discussed in the present disclosure may be implemented together in any suitable combination. Accordingly, the implementations are not to be restricted except in light of the attached claims and their equivalents. Also, various modifications and changes may be made within the scope of the attached claims.

1. A method for the production of a low-THC cannabinoid isolate comprising a target cannabinoid, the method comprising
(i) providing an initial composition comprising at least one cannabinoid, wherein said at least one cannabinoid comprises THC and said at least one target cannabinoid, which target cannabinoid is a non-THC cannabinoid and wherein a concentration of said THC is between 0.1% and 10 wt % of a total cannabinoid content of said composition;
(ii) partially separating said THC from said target cannabinoid, by a process selected to produce a first THC-enriched composition comprising a first fraction of said target cannabinoid and a first THC-depleted composition comprising a second fraction of said target cannabinoid, wherein
(a) said first THC-enriched composition comprises at least 5% wt/wt of the content of said target cannabinoid in said initial composition,
(b) a concentration of said THC wt/wt of total cannabinoids in said first THC-enriched composition is at least twice that in said initial composition, and
(c) a concentration of said THC wt/wt of total cannabinoids in said first THC-depleted composition is less than one half that in said initial composition;
(iii) selectively oxidizing at least 30% of said THC in said first THC-enriched composition to form a first oxidized composition comprising at least one oxidation product of THC, residual THC, and said target cannabinoid;
(iv) at least partially recovering said target cannabinoid from said first oxidized composition; and
(v) combining said first THC-depleted composition with at least a fraction of said target cannabinoid recovered from said first oxidized composition,
wherein said low-THC cannabinoid isolate is formed, comprising said first THC-depleted composition and said target cannabinoid recovered from said first oxidation composition.

2. The method of claim 1, wherein said recovering comprises recycling at least a fraction of said first oxidized composition for further partially separating.

3. The method of claim 1, wherein said first THC-enriched composition, said THC-depleted composition or both comprise said oxidation product.

4. The method of claim 1, wherein said low-THC cannabinoid isolate comprises said oxidation product.

5. The method of claim 3, further comprising at least partially separating said oxidation product from at least one of said THC-enriched composition and said THC-depleted composition.

6. The method of claim 1, wherein said recovering comprises blending at least a fraction of said first oxidized composition with at least a fraction of said first THC-depleted composition to form a first blend, wherein said blend is said low-THC cannabinoid isolate.

7. The method of claim 1, wherein said recovering comprises at least partially separating said residual THC from said target cannabinoid and/or said oxidation product of THC in said oxidized composition, to form a second THC-enriched composition and a second THC-depleted composition, wherein said second THC-depleted composition comprises at least 50% wt/wt of the content of said target cannabinoid in said oxidized composition.

8. The method of claim 7, wherein said recovering further comprises blending at least a fraction of said second THC-depleted composition with at least a fraction of said first THC-depleted composition to form a second blend, wherein said second blend is said low-THC cannabinoid isolate.

9. The method of claim 1, further comprising selectively oxidizing at least 30% of said THC in said second THC-enriched composition to form a second oxidized composition.

10. The method of claim 1, wherein said target cannabinoid is selected from the group consisting of cannabidiol in acid or decarboxylated form (CBDa or CBD, respectively), cannabigerol in acid or decarboxylated form (CBGa or CBG, respectively), cannabichromene in acid or decarboxylated form (CBCa or CBC, respectively), tetrahydrocannabivarin in acid or decarboxylated form (THCVa or THCV, respectively), Cannabidivarin in acid or decarboxylated form (CBDVa or CBDV respectively) and cannabinol in acid or decarboxylated form (CBNa or CBN, respectively) and combinations thereof.

11. The method of claim 1, wherein said target cannabinoid is selected from the group consisting of CBD, CBDa and combinations thereof.

12. The method of claim 1, wherein said partially separating said THC from said target cannabinoid and/or said at least partially separating said residual THC from said target cannabinoid and/or said oxidation product of THC in said oxidized composition comprises use of a method selected from the group consisting of chromatographic separation, selective adsorption, crystallization, distillation and combinations thereof,

13. The method of claim 2, wherein said separating said oxidation product from at least one of said THC-enriched composition and said THC-depleted composition comprises use of a method selected from the group consisting of chromatographic separation, selective adsorption, crystallization, distillation and combinations thereof,

14. The method of claim 1, further comprising at least partially purifying at least one low-THC composition selected from the group consisting of said first THC-depleted composition, said second THC-depleted composition, said first oxidized composition, said second oxidized composition, said first blend and said second blend to provide an increase of at least 5% in a concentration of said target cannabinoid wt/wt of a total cannabinoid content with respect to said low THC composition.

15. The method of claim 14, wherein said at least partially purifying comprises use of a method selected from the group consisting of crystallization, distillation, chromatographic separation, selective adsorption and combinations thereof.

16. The method of claim 14, wherein said at least one cannabinoid in said initial composition comprises THCA, the method further comprising at least partially decarboxylating said THCA to THC prior to or subsequent to said selectively oxidizing.
